(19) United States
(12) Patent Application Publication
(10) Pub. No.: US 2015/0252146 A1
(43) Pub. Date: Sep. 10, 2015

(54) METHOD FOR PRODUCING MODIFIED EPOXY(METH)ACRYLATE RESINS, AND THE USE THEREOF

(71) Applicant: HILTI AKTIENGESELLSCHAFT, Schaan (LI)

(72) Inventors: Gerald Gaefke, Augsburg (DE); Thomas Burgel, Landsberg am Lech (DE); Michael Leitner, Landsberg (DE)

(21) Appl. No.: 14/718,910

(22) Filed: May 21, 2015

Related U.S. Application Data
(63) Continuation of application No. PCT/EP2013/074234, filed on Nov. 20, 2013.

(30) Foreign Application Priority Data
Nov. 23, 2012 (DE) 102012221441.0

(51) Int. Cl. C08G 65/26; C07D 301/00

(52) U.S. Cl. CPC C08G 65/2684; C07D 301/00

(57) ABSTRACT

A method is described for the production of modified epoxy (meth)acrylates, wherein organic compounds containing the epoxide groups are reacted with (meth)acrylic acid in the presence of a suitable catalyst, and after at least 80% of the epoxide groups have been reacted, the product is partially reacted with the anhydride of a saturated dicarboxylic acid. The epoxy (meth)acrylates which can be obtained in this manner can be used as binders in resin mixtures and reactive resin mortar compositions, for example for the purpose of chemical fastening.

### RELATED APPLICATIONS

This application claims priority to, and is a continuation of International Patent Application No. PCT/EP2013/074234 having an International filing date of Nov. 20, 2013, which is incorporated herein by reference, and which claims priority to German Patent Application No. 10 2012 221 441.0, having a filing date of Nov. 23, 2012, which are also incorporated herein by reference in their entirety.

### FEDERALLY SPONSORED RESEARCH OR DEVELOPMENT

[Not Applicable]

### MICROFICHE/COPYRIGHT REFERENCE

[Not Applicable]

### BRIEF SUMMARY OF THE TECHNOLOGY

The invention relates to a method for the production of modified epoxy (meth)acrylates, to the epoxy (meth)acrylates produced according to this method, and to their use as free radical curable binders.

### BACKGROUND OF THE INVENTION

According to the prior art, epoxy (meth)acrylates are obtained by regioselective, ring-opening nucleophilic addition of acrylic or methacrylic acid to organic compounds which have epoxide groups, such as bisphenol A diglycidyl ether, in the presence of suitable basic catalysts, for example heteroaromatic nitrogen compounds (for example, DE-P-4004091). Monomers which can be used immediately and which can be polymerized by free-radical initiators are obtained as a result of this process, with no after-treatment.

A disadvantage of binders based on these monomers for use in mortar compositions used for chemical fastening is their relatively low bond strength, low degree of three-dimensional cross-linking, high shrinkage, and the inhibition of surface curing by oxygen. The high residual double bond content after crosslinking polymerization is further disadvantageous, with the result that it is not possible to achieve high bond strength.

Accordingly, additional compounds are employed in chemical fastening systems with a binder based on epoxy (meth)acrylates, which counteract the disadvantages mentioned above. In order to achieve high bond strengths as well as good three-dimensional cross-linking, co-polymerizable monomers are used which have either two free-radical polymerizable end groups or one free-radical polymerizable end group and one polar end group, such as a hydroxyl-group. Hydroxyalkyl (meth)acrylates are often used for this purpose. However, these have the disadvantage that some of these are hazardous to health and therefore subject to labeling requirements—such as the commonly-used hydroxypropyl methacrylate (HPMA), which is required to be labeled as an irritant. As a result, reactive resin systems containing these compounds in a certain amount are also subject to labeling requirements.

Another approach has been focused on reducing the free hydroxyl-groups and therefore reducing the hydrophilic groups. For the purpose of modifying the addition product of bisphenol A diglycidyl ether and methacrylic acid, called Bis-GMA, the hydroxyl-groups thereof have been reacted with methacrylic acid (U.S. Pat. No. 4,357,456), methacryloyl chloride (U.S. Pat. No. 3,721,644), lactones (DE 33 34 329), succinic anhydride (DE-P-2610146), or diisocyanates (U.S. Pat. No. 3,629,187). However, long reaction times and relatively high temperatures are required for these reactions, thereby making the risk of premature polymerization of Bis-GMA during the modification extremely high. Because—as is known—the network polymers of the unmodified Bis-GMA already have a relatively high content of residual double bonds, additional double bonds in the molecule—as suggested in U.S. Pat. No. 4,357,456 and U.S. Pat. No. 3,721,644—are rather disadvantageous.

When Bis-GMA is reacted with lactones, and/or the dibenzoate of bisphenol A diglycidyl ether is reacted with glycidyl (meth)acrylate, hydroxyl-groups are formed again—meaning that the hydrophilicity of the molecule will remain unchanged.

It is also a disadvantage in this modification that, in the method according to DE 4109048 A1, polyadducts are formed by reaction of the two hydroxyl-groups of the Bis-GMA with bifunctional compounds such as succinic anhydride, resulting in greatly increased viscosity of the modified epoxy (meth)acrylate, and therefore greater difficulty in later processing.

As a result, there is a need for a simple method in which no purification or separation of the final product is required, such that the reaction mixture can be used directly, and in which there is no premature polymerization of the reactants or products.

### BRIEF SUMMARY OF THE INVENTION

In one embodiment, the method for the production of modified epoxy (meth)acrylates, comprises reacting organic compounds containing epoxide groups with (meth)acrylic acid in the presence of a suitable catalyst, and after at least 80% of the epoxide groups have been reacted, partially reacting a product from the reaction with a anhydride of a saturated dicarboxylic acid.

The epoxy (meth)acrylate resin made in the above method can be used as a binder in free radical curable resin mixtures or as a binder in free radical curable reactive resin mortar compositions. Both binders can be used for chemical fastening.

### BRIEF DESCRIPTION OF SEVERAL VIEWS OF THE DRAWINGS

[Not Applicable]

### DETAILED DESCRIPTION OF THE INVENTION

The problem addressed by the invention is that of providing a method for the production of modified addition products of acrylic or methacrylic acid and organic compounds which contain epoxide groups, said method being easy to carry out and to control.

This problem is addressed according to the invention by the provision of a method for the production of modified epoxy (meth)acrylates, wherein organic compounds having epoxide groups are reacted with (meth)acrylic acid, and once at least 80% of the epoxide groups have been reacted the product is reacted with the anhydride of a saturated dicarboxylic acid. Accordingly, the modified epoxy (meth)acrylates are produced in a two-step, one-pot reaction, resulting in a shelf-stable product which can be immediately employed for all applications.

The following explanations of terminology used in the context of the invention are included here as practical assistance to understanding the invention:
“base resin”: the pure, curing or curable compound which is cured by polymerization alone or with reagents such as curing agents, accelerators, and the like (not included in the base resin); the curable compounds can be monomers, dimers, oligomers and prepolymers;
“resin master batch”: the product of the manufacture of the base resin following synthesis (without isolation of the base resin), which can contain reactive diluents, stabilizers and catalysts;
“resin mixture”: a mixture of the resin master batch and accelerators, as well as stabilizers and optionally other reactive diluents; this term is used interchangeably with the term “organic binder”;
“reactive resin mortar”: a mixture of the resin mixture and inorganic aggregates; the term “A component” is used interchangeably;
“curing agents”: substances which cause the polymerization (curing) of the base resin;
“hardener”: a mixture of curing agent and organic and/or inorganic aggregates;
“accelerator”: a compound which is capable of speeding up the polymerization reaction (curing), which serves to accelerate the formation of the free radical initiator;
“polymerization inhibitor” a compound capable of inhibiting the polymerization reaction (curing), which serves, on the one hand, to prevent the polymerization reaction and therefore an undesired premature polymerization of the free radical polymerizable compound during storage—wherein these compounds are typically used in such small amounts that the gel time is not affected; or on the other hand, the polymerization inhibitor serves the purpose of delaying the polymerization reaction immediately after the addition of the curing agent, wherein the compounds are usually used in amounts such that the gel time is affected;
“reactive diluent”: liquid or low-viscosity base resins which dilute other base resins, the resin master batch, or the resin mixture, thereby providing the necessary viscosity for the application thereof, which contain functional groups capable of reacting with the base resin, and which become the majority component of the cured composition (mortar) in the polymerization (curing); also referred to as co-polymerizable monomer.
“gel time”: For unsaturated polyester or vinyl resins, which are usually cured with peroxides, the duration of the curing phase of the resin corresponds to the gel time, during which the temperature of the resin increases from +25° C. to +35° C. This corresponds roughly to the period in which the fluidity or viscosity of the resin is still in such a range that the reactive resin or the reactive resin composition can be easily processed and/or finished;
“mortar composition”: a formulation which contains, in addition to the reactive resin composition, additional organic or inorganic fillers and can be used directly for the purpose of chemical fastening without further preparation;
“(meth)acryl . . . / . . . (meth)acryl . . . ”: used to denote both “methacryl . . . / . . . methacryl . . . ” compounds and “acryl . . . / . . . acryl . . . ” compounds;
“epoxy (meth)acrylates”: derivatives of epoxide resins which have acrylate- or methacrylate groups and which are substantially free of epoxy groups;
“epoxide equivalent weight”: the amount of epoxy resin in [g] which comprises and functions as one epoxide equivalent [eq]: the epoxide equivalent weight is calculated from the molar mass M in [g/mol] divided by the functionality f in [eq/mol]: (EEW [g/eq]);
“carboxylic acid equivalent weight”: the amount of carboxylic acid compound in [g] which comprises and functions as one carboxylic acid equivalent [eq], and is calculated from the molar mass M in [g/mol] divided by the functionality f in [eq/mol]; (COOH-EW [g/eq]);
“cold-curing”: means that the resin mixtures and reactive resin mortar can completely cure at room temperature.

As the organic compounds which contain epoxide groups, it is advantageous that those which have a molecular weight corresponding to a number average molecular mass 3 in the range from 129 to 2400 g/mol, and which contain on average at least one, and preferably 1.5 to 2 epoxide groups per molecule, are used. Particularly preferred are the epoxide groups of the glycidyl ether or glycidyl ester type, obtained by reacting an epihalohydrin, particularly epichlorohydrin, with a mono- or multi-functional aliphatic or aromatic hydroxyl-compound, thiol-compound, carboxylic acid, or a mixture thereof. The resulting organic compounds containing epoxide groups have an epoxide equivalent weight (EEW) which is preferably in the range from 87 to 1600 g/eq, more preferably in the range of 160 to 800 g/eq, and most preferably in the range of 300 to 600 g/eq.

Examples of suitable compounds which contain epoxide groups, are—but are not restricted to—polyglycidyl ethers of polyhydric phenols such as pyrocatechol, resorcinol, hydroquinone, 4,4′-dihydroxydiphenyl methane, 2,2-(4,4′-dihydroxydiphenyl) propane (bisphenol A), bis(4-hydroxyphenyl) methane (bisphenol F), 4,4′-dihydroxydiphenylsulfone (bisphenol S), 4,4′-dihydroxydiphenyl cyclohexane, tris(4-hydroxyphenyl) methane, and novolacs (i.e., from reaction products of monohydric or polyhydric phenols with aldehydes, particularly formaldehyde, in the presence of acid catalysts) such as phenol novolac resin and cresol novolac resin.

In addition, the following are named by way of example, but not as an exhaustive list: glycidyl ethers of monohydric alcohols such as n-butanol or 2-ethylhexanol; or glycidyl ethers of polyhydric alcohols such as 1,4-butanediol, 1,4-butanediol, 1,6-hexanediol, glycerol, benzyl alcohol, neopentyl glycol, ethylene glycol, cyclohexane dimethanol, trimethylolpropane, pentaerythritol and polyethylene glycols, triglycidyl isocyanurate; polyglycidyl polyhydric thiols such as bis(mercaptomethyl)benzol; or glycidyl esters of monocarboxylic acids such as versatic acid; or glycidyl esters of polybasic, aromatic and aliphatic carboxylic acids, such as diglycidyl ester of phthalic acid, isophthalic diglycidyl ester, terephthalic diglycidyl ester, tetrahydrophthalic diglycidyl ester, adipic acid diglycidyl ester and hexahydrophthalic diglycidyl ester.

Diglycidyl ethers of dibasic hydroxyl-compounds of the general formula (I) are particularly preferred as organic compounds containing epoxide groups:

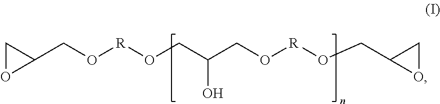

wherein R is an unsubstituted or substituted aliphatic or aromatic group, preferably an aromatic group, and more preferably an aromatic group having 6 to 24 carbon atoms, wherein the average value for n is 0 to 3. R is particularly preferably a group of the bisphenol type, such as bisphenol A, bisphenol F or bisphenol S, or of the novolac type, wherein a bisphenol-type group is very particularly preferred. n is preferably approximately 0.1, approximately 1, or approximately 2. In the context of the invention, compounds in which n is ≈0.1 are considered as monomers, and compounds in which n is ≈1 or 2 are considered as polymers.

The epoxy (meth)acrylate resins are obtained in a first step (i) by the reaction of an organic compound containing an epoxide group with acrylic acid or methacrylic acid, such that the resins necessarily have acryloxy- or methacryloxy-groups in terminal positions, and hydroxyl-groups at the 2-position relative to the established acryloxy- or methacryloxy-group (also called β-hydroxyl-groups below) in the primary chain of the molecule. 0.7 to 1.2 carboxylic acid equivalents of (meth)acrylic acid are advantageously used per equivalent of epoxide. The organic compounds which contain epoxide groups, and the (meth)acrylic acid, are preferably used in approximately stoichiometric ratios in this case—that is, per epoxide equivalent of the organic compound, about one equivalent of (meth)acrylic acid is used.

The reaction of the organic compound which has epoxide groups with the (meth)acrylic acid is carried out in the known manner by joining the components.

The reaction is carried out either neat or in suitable solvents. Suitable solvents are, by way of example, inert solvents such as butyl acetate, toluene, cyclohexane, or mixtures of such solvents, monomers of the glycidyl ester or glycidyl ether type (type I reactive diluents) or copolymerizable monomers (type II reactive diluents), which are named below as examples. It is preferred that no solvent is used, but if necessary, type I or type II reactive diluents are used, wherein type II reactive diluents are more strongly preferred. The inert solvents function in the cured resin and/or composition as plasticizers, such that their use is strongly dependent on the use of the cured product and/or composition.

Suitable type I reactive diluents are allyl glycidyl ether, butyl glycidyl ether (BGE), 2-ethylhexyl glycidyl ether, alkyl glycidyl ethers (C12-C14), tridecyl glycidyl ether, phenyl glycidyl ether (PGE), o-cresol glycidyl ether (CGE), p-tert-butyl glycidyl ether, resorcinol diglycidyl ether (RDGE), 1,4-butanediol diglycidyl ether (BDGE), 1,6-hexanediol diglycidyl ether (HDGE), cyclohexanedimethanol diglycidyl ether, neopentyl glycol diglycidyl ether, trimethylolpropane triglycidyl ether, glycerol triglycidyl ether, polypropylene glycol diglycidyl ether, and epoxidized vegetable oils such as epoxidized linseed oil and epoxidized castor oil.

The organic compound containing epoxide groups, the (meth)acrylic acid, and optionally the solvent are added to a reaction vessel at room temperature, in particular at 10° C. to 40° C., and mixed, preferably with stirring.

The reaction is then carried out at approx. +80° C. to +120° C. It is preferred, especially in the case of a compound with epoxide groups which has a high molecular weight, that the mixture is heated as fast as possible to this temperature in order to prevent sticking of the mixture to the reaction vessel or the stirrer. The heating preferably occurs over a period of between 30 minutes and 3 hours, depending on the amount present—wherein this time can vary greatly depending on the molecular weight of the compound which has the epoxide groups. For higher molecular weights, a shorter period is better, and for lower molecular weights, a longer period is a possibility.

The epoxide group-containing compound is reacted with the (meth)acrylic acid in the presence of about 0.01 to 3 wt % of a suitable catalyst, with respect to the compound which contains the epoxide groups. Suitable catalysts are, for example, tertiary amines, quaternary ammonium salts, alkali hydroxides, alkali salts of organic carboxylic acids, mercaptans, dialkyl sulphides, sulphonium compounds, phosphonium compounds, or phosphines. Quaternary ammonium salts such as tetraethylammonium chloride, tetraethylammonium bromide, tetrabutylammonium chloride, tetrabutylammonium bromide, and the like are preferably used.

The catalyst is added to the mixture along with the (meth)acrylic acid once the reaction temperature has been reached.

Alternatively, it is possible to add the catalyst and the (meth)acrylic acid, either partially or completely as a mixture, to the reaction vessel along with the other compounds. It is also possible to add the catalyst or the (meth)acrylic acid to the reaction vessel together with the other compounds, then to add the other components after heating to the reaction temperature.

To stabilize against premature polymerization of the polymerizable reaction products produced according to the invention, and of the optionally added type II reactive diluents, during the entire production process and during the storage of the reaction product, it is advantageous to add at least 0.0005 to 2 wt %, with respect to the entire resin mixture, including any auxiliary agents and additives, of at least one suitable polymerization inhibitor prior to the reaction. However, the at least one polymerization inhibitor can also be added during or following the reaction. The polymerization inhibitor can, if necessary, be added in a volume of 2 wt %, preferably 0.01 to 1 wt %, with respect to the entire reaction mixture.

As polymerization inhibitors, polymerization inhibitors commonly used for free radical polymerizable compounds, known to a person skilled in the art, are suitable according to the invention.

To stabilize against premature polymerization, resin mixtures and reactive resin mortars typically contain polymerization inhibitors such as hydroquinone, substituted hydroquinones, e.g. 4-methoxyphenol, phenothiazine, benzoquinone or tert-butylcatechol, as described in EP 1935860 A1 or EP 0965619 A1, for example, stable nitroxyl-radicals, also called N-oxyl-radicals, such as piperidinyl-N-oxyl or tetrahydropyrrolidine-N-oxyl, as described in DE 19531649 A1. It is particularly preferred that 4-hydroxy-2,2,6,6-tetramethylpiperidine-N-oxyl (referred to as Tempol in the following) is used for stabilization, which offers the advantage that it is also possible to adjust the gel time by means of the same.

The polymerization inhibitors are preferably chosen from among phenolic compounds and non-phenolic compounds, such as stable free radicals and/or phenothiazines.

As phenolic polymerization inhibitors, which are often components of commercial free radical curing reactive resins, phenols such as 2-methoxyphenol, 4-methoxyphenol, 2,6-Di-tert-butyl-4-methylphenol, 2,4-Di-tert-butylphenol, 2,6-Di-tert-butylphenol, 2,4,6-trimethylphenol, 2,4,6-tris(dimethylaminomethyl) phenol, 4,4′-thio-bis(3-methyl-6-tert-butylphenol), 4,4′-isopropylidenediphenol, 6,6′-Di-tert-butyl-4,4′-bis(2,6-Di-tert-butylphenol), 1,3,5-trimethyl-2,4,6-tris(3,5-Di-tert-butyl-4-hydroxybenzyl)benzene, 2,2′-methylene-di-p-cresol, pyrocatechol and butylpyrocatechols such as 4-tert-butylcatechol, 4,6-Di-tert-butylcatechol, hydroquinones such as hydroquinone, 2-methylhydroquinone, 2-tert-butylhydroquinone, 2,5-Di-tert-butylhydroquinone, 2,6-Di-tert-butylhydroquinone, 2,6-dimethylhydroquinone, 2,3,5-trimethylhydroquinone, benzoquinone, 2,3,5,6-tetrachloro-1,4-benzoquinone, methylbenzoquinone, 2,6-dimethylbenzoquinone, naphthoquinone, or mixtures of two or more thereof, can be contemplated.

As non-phenolic polymerization inhibitors, the following are preferred: phenothiazines such as phenothiazine and/or derivatives or combinations thereof, or stable organic free radicals such as galvinoxyl and N-oxyl radicals.

Suitable stable N-oxyl radicals (nitroxyl radicals) can be selected from among 1-oxyl-2,2,6,6-tetramethylpiperidine, 1-oxyl-2,2,6,6-tetramethylpiperidin-4-ol (also referred to as TEMPOL), 1-oxyl-2,2,6,6-tetramethylpiperidin-4-one (also referred to as TEMPON), 1-oxyl-2,2,6,6-tetramethyl-4-carboxyl-piperidine (also referred to as 4-carboxy-TEMPO), 1-oxyl-2,2,5,5-tetramethylpyrrolidine, 1-oxyl-2,2,5,5-tetramethyl-3-carboxylpyrrolidin (also referred to as 3-carboxy-PROXYL), aluminum-N-nitrosophenyl hydroxylamine, and diethylhydroxyl amine, as described in DE 199 56 509. Additional suitable N-oxyl compounds are oximes such as acetaldoxime, acetone oxime, methyl ethyl ketoxime, salicyloxime, benzoxime, glyoximes, dimethylglyoxime, acetone-O-(benzyloxycarbonyl) oxime and the like. Furthermore, pyrimidinol derivatives or pyridinol compounds which are substituted in the para-position to the hydroxyl-group can be used as polymerization inhibitors, as described in the previously unpublished patent DE 10 2011 077 248 B1.

The polymerization inhibitors can be used, depending on the desired properties and the application for the resin mixture, either alone or as a combination of two or more of the same. The combination of the phenolic and the non-phenolic polymerization inhibitors enables a synergistic effect in this case, which is also shown by the adjustment of a substantially drift-free adjustment [sic] of the gel time of the reactive resin formulation.

According to the invention, the reaction of the organic compound which contains the epoxide groups with the (meth)acrylic acid is continued until at least 80%/o, preferably at least 90%, and more preferably at least 95%, of the epoxide groups have been reacted.

The conversion of the epoxide groups is continuously determined during the reaction by titration of the epoxide groups according to DIN 16945.

The modified epoxy (meth)acrylate resins are obtained according to the invention by esterification of a part of the β-hydroxyl-groups of the epoxy (meth)acrylate, the same formed during the reaction of the organic compound containing the epoxide groups with (meth)acrylic acid, with the anhydride of a saturated C3-C5-dicarboxylic acid. The saturated C3-C5-dicarboxylic acid is selected from among propanedioic acid (also: malonic acid), succinic acid, and pentanedioic acid (also: glutaric acid). The succinic anhydride is particularly preferred according to the invention.

The inventors have discovered that the esterification of the β-hydroxyl-groups only proceeds to completion with the anhydrides at the selected reaction temperature. The free oxygen group formed during the esterification does not react at these temperatures—or only to a very small degree which can be ignored. Where dicarboxylic acids are used, it is necessary to select higher reaction temperatures in order to even achieve esterification at all. However, these temperatures are then so high that it is no longer possible to ensure a selective reaction of only one oxygen group of the dicarboxylic acid, and both oxygen groups react—at least to a large degree—resulting in undesired cross-linking at this point.

The esterification reaction is likewise carried out at approx. +80° C. to +120° C., wherein the anhydride is added directly to the reaction mixture of the reaction of the organic compound containing the epoxide groups and the (meth)acrylic acid without isolating the resulting products. 1 to 50 mol % (mol %/OH), preferably 2 to 30 mol %, and more preferably 3 to 15 mol % (mol %/OH) of the dicarboxylic acid anhydride is used per β-hydroxyl-group of the epoxy (meth)acrylate formed in the reaction of the organic compound containing the epoxide groups and the (meth)acrylic acid.

After the dicarboxylic acid anhydride is added, the reaction mixture is held at the reaction temperature of +80° C. to +120° C. for a period of six hours. After the reaction has finished, the reaction mixture is cooled to room temperature.

The reaction products produced according to the invention can be used without the addition of solvents. They can also optionally be diluted with type II reactive diluents in order to adjust the desired viscosity.

Suitable type II reactive diluents are described in the applications EP 1 935 860 A1 and DE 195 31 649 A1. The resin mixture preferably contains a (meth)acrylic acid ester as a reactive diluent, wherein it is particularly preferred that aliphatic or aromatic C5-C15-(meth)acrylates are selected. Suitable examples include: hydroxypropyl (meth)acrylate, 1,2-ethanediol di(meth)acrylate, 1,3-propanediol di(meth)acrylate, 1,3-butanediol di(meth)acrylate, 1,4-butanediol di(meth)acrylate, trimethylolpropane tri(meth)acrylate, phenethyl (meth)acrylate, tetrahydrofurfuryl (meth)acrylate, ethyltriglycol (meth)acrylate, N,N-dimethylaminoethyl (meth)acrylate, N,N-dimethylaminomethyl (meth)acrylate, acetoacetoxyethyl (meth)acrylate, isobornyl (meth)acrylate, 2-ethylhexyl (meth)acrylate, diethylene glycol di(meth)acrylate, methoxypolyethylene (meth)acrylate, trimethylcyclohexyl (meth)acrylate, 2-hydroxyethyl (meth)acrylate, dicyclopentenyloxyethyl (meth)acrylate and/or tricyclopentadienyl di(meth)acrylate, bisphenols A (meth)acrylate, novolac epoxy di(meth)acrylate, di-[(meth)acryloyl-maleoyl]tricyclo-[5.2.1.0.2.6]decane, dicyclopentenyl oxyethyl crotonate, 3-(meth)acryloyl oxymethyl tricylo-[5.2.1.0 2.6.]decane, 3-(metha)cyclopentadienyl (meth)acrylate, isobomyl (meth)acrylate, and decalyl-2-(meth)acrylate; PEG di(meth)acrylates such as PEG200 di(meth)acrylate, tetraethylene glycol di(meth)acrylate, solketal (meth)acrylate, cyclohexyl (meth)acrylate, phenoxyethyl di(meth)acrylate, methoxyethyl (meth)acrylate, tetrahydrofurfuryl (meth)acrylate, tert-butyl (meth)acrylate and norbornyl (meth)acrylate. In principle, other conventional free-radical polymerizable compounds can be used, either alone or in a mixture with the (meth)acrylic acid esters, including styrene, α-methylstyrene, alkylated styrenes such as tert-butyl styrene, divinyl benzene and allyl compounds, for example, wherein the non-hazardous representatives thereof are preferred.

The products produced according to the invention constitute valuable systems which can be cured by means of suitable substances which supply free radicals, such as (hydro)peroxides, optionally in the presence of accelerators.

The products produced according to the invention are preferably used as binder components for glues, adhesive agents, sealing agents, and coating agents. The products produced according to the invention are particularly preferably used as binders for free radical curable—particularly cold curing—mortar compositions for the purpose of chemical fastening.

Therefore, a further object of the invention is the use of the epoxy (meth)acrylate resin produced according to the invention as a binder in free radical curable resin mixtures, as well as reactive resin mortar compositions containing this resin mixture, particularly for chemical fastening.

Reactive resin mortars are generally produced by adding the starting compounds which are necessary for the production of the base resin, optionally together with catalysts and solvents, particularly reactive diluents, into a reactor, and initiating reaction among them. After the reaction is complete, and optionally at the beginning of the reaction, polymerization inhibitors are added to the reaction mixture to increase shelf life, thereby producing the so-called resin master batch. Accelerators for the curing of the base resin, optionally additional polymerization inhibitors if necessary—to adjust the gel time, wherein the same can be identical to or different from the stabilizer used for storage stability—and optionally further solvents, particularly reactive diluents, are frequently added to the resin master batch, thereby producing the resin mixture. For the adjustment of the gel time and the reactivity, a further 0.005 to 3 wt %, and preferably 0.05 to 1 wt %, with respect to the resin mixture, of a polymerization inhibitor can be included. For the purpose of adjusting various properties such as the rheology and the concentration of the base resin, inorganic and/or organic aggregates are added to this resin mixture, thereby producing the reactive resin mortar.

A preferred resin mixture accordingly contains at least one base resin, at least one reactive diluent, at least one accelerator, and at least one polymerization inhibitor. A reactive resin mortar contains, in addition to the resin mixture just described, organic and/or inorganic aggregates, wherein inorganic aggregates are particularly preferred.

The method according to the invention is explained in greater detail in the following examples, which do not restrict the invention to their subject matter.

### EMBODIMENTS

### A) Resin Master Batch Syntheses

### Example 1

### Monomer Resin, n˜1

223 g of bisphenol A diglycidyl ether (EEW (DIN 16945), 182-192 g/eq; Epilox® A 19-03; LEUNA-Harze GmbH) is filled in its entirety into the reactor at room temperature, then 110 g of methacrylic acid, 0.1 g of phenothiazine, and 2 g of tetraethyl ammonium bromide are added. The reaction mixture is heated on a linear heating curve to approx. 80° C. over 30 minutes, and held at this temperature for 20 hours.

The conversion of the epoxide groups is determined continuously during the reaction by titration of the epoxy groups according to DIN 16945.

Once a conversion of at least 97% is achieved, 20 mol %/OH of succinic anhydride is added, and stirring proceeds at a temperature of 80° C. Following a reaction time of 6 hours, the reaction mixture is cooled to room temperature. The result is a resin master batch which is ready for use.

### Example 2

### Polymer Resin, n˜1

273 g of bisphenol A diglycidyl ether (EEW (DIN 16945) 300-340 g/eq; Epilox® A 32-02; LEUNA-Harze GmbH) is filled in its entirety into the reactor at room temperature, to which is added 88 g PEG200 dimethacrylate, 79 g methacrylic acid, 0.1 g of phenothiazine, and 3 g of tetraethyl ammonium bromide. The reaction mixture is heated on a linear heating curve to approx. 80° C. over 30 minutes, and held at this temperature for 20 hours.

The conversion of the epoxide groups is determined continuously during the reaction by titration of the epoxy groups according to DIN 16945.

Once a conversion of at least 97% is achieved, 10 mol %/OH of succinic anhydride is added, and stirring proceeds at a temperature of 80° C. Following a reaction time of 6 hours, the reaction mixture is cooled to room temperature. The result is a resin master batch which is ready for use.

### Example 3

### Polymer Resin, n˜2

324 g of bisphenol A diglycidyl ether (EEW (DIN 16945), 450-500 g/eq; Epilox® A 50-02; LEUNA-Harze GmbH) is filled in its entirety into the reactor at room temperature, to which is added 97 g of PEG200 dimethacrylate, 63 g of methacrylic acid, 0.04 g of phenothiazine, 0.08 g of 4-hydroxy-2,2,6,6-tetramethylpiperidinyl-N-oxyl and 3 g of tetraethyl ammonium bromide. The reaction mixture is heated on a linear heating curve to approx. 100° C. over 30 minutes, and held at this temperature for 5 hours.

The conversion of the epoxide groups is determined continuously during the reaction by titration of the epoxy groups according to DIN 16945.

Once a conversion of at least 97% is achieved, 10 mol %/OH of succinic anhydride is added, and stirring proceeds at a temperature of 100° C. Following

a reaction time of 2 hours, the reaction mixture is cooled to room temperature. The result is a resin master batch which is ready for use.

### B) Resin Mixtures

For the preparation of the resin mixtures, each of the resin master batches A to C described above is mixed with PEG200DMA, 1,4-butanediol dimethacrylate (BDDMA), tert-butyl pyrocatechol (tBBK), and 4-hydroxy-2,2,6,6-tetramethylpiperidine-1-oxyl (Tempol). The amounts used are listed in Table 1 below. Subsequently, the gel time of each resulting resin mixture is adjusted by means of an aromatic amine to approx. 6 minutes.

TABLE 1
Amounts of the components for the preparation of the resin mixtures

| | A1/B1/C1 | A2/B2 | A3/B3/C3 |
|---|---|---|---|
| | (each n~0.1) | (each n~1) | (each n~2) |
| Component | Amount [wt %] | | |
| Resin master batch | 39.2 | 42.54 | 36.14 |
| PEG200DMA | 25.4 | 19.6 | 23.5 |
| BDDMA | 35.3 | 37.8 | 40.3 |
| tBBK | 0.05 | 0.04 | 0.04 |
| Tempol | 0.015 | 0.015 | 0.015 |

The gel time is determined by means of a commercially available device (GELNORM® gel timer) at a temperature of 25° C. For this purpose, each of the A and the B components are mixed at a volume ratio of 3:1, and heated immediately after mixing in a silicone bath to 25° C., whereupon the temperature of the sample is measured. The sample itself is situated in a test tube which is placed in an air jacket lowered into a silicone bath for the heating process.

The heat generation of the sample is plotted against time. The evaluation is made according to DIN 16945, Part 1 and DIN 16916. The gel time is the time at which a temperature rise of 10 K is achieved—in this case from 25° C. to 35° C.

The ready-for-use, shelf-stable resin mixture is obtained in this way.

### C) Reactive Resin Mortar

To produce the hybrid resin, the resin mixtures are mixed to a homogenous mortar composition in a dissolver with 30-45 parts by weight of silica sand, 15-25 parts by weight of cement, and 1-5 parts by weight of fumed silica.

### D) Hardener Component

To produce the hardener component, 13 g of dibenzoyl peroxide, 23 g of water, 1 g of fumed silica, 19 g of alumina and 46 g of quartz powder with a suitable particle size distribution are mixed in a dissolver to form a homogeneous composition.

### Determination of the Bond Stress Failure (τ)

M12 threaded anchor rods are used to determine the bond stress failure of the cured material, said anchor rods being inserted, with the reactive resin mortar compositions in the examples and the comparative examples, into bore holes in concrete which have a diameter of 14 mm and a hole depth of 72 mm. The average failure loads are determined by centered tension on the threaded anchor rods. In each case, three threaded anchor rods are anchored in bore holes, and their load values are determined after 24 h of hardening. The bond strengths r determined in this case (N/mm2) are reported below in Table 2 as averages.

Various bore hole conditions and/or curing conditions were tested as listed below.

| Test condition | Note |
|---|---|
| Reference | thoroughly cleaned, hammer-drilled hole, curing at room |
| | temperature (20° C.) |
| −10° C. | Reference holes, setting and curing at a substrate |
| | temperature of −10° C. |
| +40° C. | Reference holes, setting and curing at a substrate |
| | temperature of +40° C. |

TABLE 2
Bond strengths (τ) of the modified Bis-GMA resins

| | | Examples | | |
|---|---|---|---|---|
| | | 1 | 2 | 3 |
| | | (n~0.1) | (n~1) | (n~2) |
| Bond strengths τ [N/mm²] | Ref | 21.5 ± 0.5 | 20.0 ± 1.6 | 18.0 ± 2.0 |
| | −10° C. | 18.0 ± 1.6 | 16.8 ± 1.3 | 13.6 ± 1.2 |
| | +40° C. | 21.4 ± 1.2 | 20.7 ± 1.6 | 19.7 ± 0.9 |

1. A method for the production of modified epoxy (meth)acrylates, comprising
reacting organic compounds containing epoxide groups with (meth)acrylic acid in the presence of a suitable catalyst, and
after at least 80% of the epoxide groups have been reacted, partially reacting a product from the reaction with a anhydride of a saturated dicarboxylic acid.

2. A method according to claim 1 wherein the organic compounds containing epoxide groups have a number average molar mass in the range from 129 to 2400 g/mol.

3. A method according to claim 2 wherein the organic compounds containing epoxide groups have an epoxide equivalent weight (EEW) in the range from 87 to 1600 g/eq.

4. A method according to claim 1 wherein 0.7 to 1.2 carboxylic acid equivalents of (meth)acrylic acid are used per epoxide equivalent.

5. A method according to claim 1 wherein between 1 and 50 mol % of the anhydride of a saturated dicarboxylic acid is used per β-hydroxyl-group of the epoxy (meth)acrylate formed during the reaction of the organic compound containing the epoxide groups with the (meth)acrylic acid.

6. A method according to claim 1 wherein the dicarboxylic acid is a C₃-C₅-dicarboxylic acid.

7. A method according to claim 1 wherein the reaction of the organic compound containing the epoxide groups with the (meth)acrylic acid is carried out in the presence of a co-polymerizable monomer.

8. A method according to claim 1 wherein the reaction of the organic compound containing the epoxide groups with the (meth)acrylic acid is carried out in the presence of a polymerization inhibitor.

9. An epoxy (meth)acrylate resin obtained by
reacting organic compounds containing epoxide groups with (meth)acrylic acid in the presence of a suitable catalyst, and
after at least 80% of the epoxide groups have been reacted, partially reacting a product from the reaction with a anhydride of a saturated dicarboxylic acid.

10. The use of the epoxy (meth)acrylate resin according to claim 9 as a binder in free radical curable resin mixtures.

11. The use of the epoxy (meth)acrylate resin according to claim 9 as a binder in free radical curable reactive resin mortar compositions.

12. The use of the resin mixture according to claim 10 for chemical fastening.

13. The use of the reactive resin mortar composition according to claim 11 for chemical fastening.
